(19) United States
(12) Patent Application Publication
(10) Pub. No.: US 2026/0240752 A1
(43) Pub. Date: Aug. 20, 2026

(54) EXFOLIATING COMPOSITION

(71) Applicant: L'OREAL, PARIS (FR)

(72) Inventors: ESTELLE MATHONNEAU, SAINT-OUEN (FR); BÉATRICE BINUTTI, CHEVILLY LARUE (FR); MAUD RIBADEAU DUMAS, CHEVILLY LARUE (FR); CLÉMENCE MACIEJEWSKI, CHEVILLY LARUE (FR)

(73) Assignee: L'OREAL, Paris (FR)

(21) Appl. No.: 19/160,990

(22) PCT Filed: Mar. 1, 2024
(86) PCT No.: PCT/EP2024/055466

§ 371 Date: Aug. 29, 2025

(30) Foreign Application Priority Data
Mar. 1, 2023 (FR) FR2301921

(51) Int. Cl. A61K 8/96; A61K 8/02; A61K 8/73; A61Q 19/10

(52) U.S. Cl. CPC A61K 8/965; A61K 8/022; A61K 8/731; A61Q 19/10; A61K 2800/28

(57) ABSTRACT

The present invention relates to an exfoliating and/or scrubbing cosmetic composition for keratin materials, in particular the skin, comprising a combination of perlite, pumice stone, microcrystalline cellulose, and cellulose, wherein the weight ratio of pumice stone to microcrystalline cellulose is greater than or equal to 1, preferably greater than 1. It also relates to the cosmetic use of a composition according to the invention, as a cleansing product and/or as an exfoliating product and/or as a care mask.

### TECHNICAL FIELD

The invention relates to an exfoliating cosmetic composition which can be used for scrubbing and/or deeply cleansing the skin of the face, neck and/or body of a human being. In particular, this composition allows the very gentle removal of dead cells and impurities from the skin.

### PRIOR ART

The aim of exfoliating or scrubbing products, also known as “scrubs”, is to make the skin of the face, body and/or neck softer, and better prepared for the application of a product, in particular a care product, a makeup product or a self-tanning product. They are generally in the form of emulsions which contain powder of polyethylene, fine particles of quartz, plant kernels or seeds (nutshells, for example) as exfoliating particles. These particles generally have a number-median diameter D50 ranging from 300 μm to 800 μm.

Unfortunately, because of the hard nature, their more or less controlled shape, and/or the very size of the exfoliating particles used, these known exfoliating products generally have the disadvantage of being abrasive, irritating and poorly tolerated by sensitive skin, and the most fragile and thin skin such as that around the eyes. In addition, these conventional products can cause drying of the skin, in particular as a result of excessive removal of the protective hydrolipidic barrier of the epidermis, thereby requiring, after their use, the application of a nutritive, protective and/or moisturizing cream. Moreover, despite all the precautions taken during the application of these formulas to avoid the area around the eyes, problems of eye intolerance have also been observed in certain cases.

### DISCLOSURE OF THE INVENTION

There is therefore still a need for a new scrub composition (or exfoliating or scrubbing composition) which is well tolerated by even sensitive skin, which does not present any ophthalmic risk, while at the same time allowing optimum scrub effectiveness, and which does not require additional treatment of the skin after scrubbing, nor does it cause the skin to dry out.

### SUMMARY OF THE INVENTION

A subject of the invention is therefore the cosmetic use of a combination of perlite, pumice stone, microcrystalline cellulose, and cellulose as an exfoliating and/or scrubbing and/or cleansing agent for keratin materials, notably the skin, in particular the face, neck, body and/or scalp.

A subject of the invention is in particular a cosmetic composition, in particular a cleansing and/or exfoliating and/or scrubbing and/or massaging composition for keratin materials, in particular the skin, characterized in that it comprises, in a cosmetically and/or dermatologically acceptable medium: a combination of perlite, pumice stone, microcrystalline cellulose, and cellulose.

The inventors have shown that the use, in an exfoliating composition, of a particular combination of several exfoliating filler(s), in particular complying with certain criteria in terms of weight ratio, or even of specific shape, and/or of particle size, makes it possible to obtain: an effective scrub, a deep cleansing, a soft skin, a smoothing effect, a brighter/lighter/more radiant complexion, while being particularly gentle, and respecting the skin sufficiently so as not to require the application of a restorative or relipidizing care product following the scrubbing or exfoliation process.

In the context of the present invention, and unless otherwise indicated, the following definitions apply:

According to the invention, the term “keratin materials” is intended to denote the skin of the body, of the face and/or of the area around the eyes, the lips, the nails, the mucous membranes, the eyelashes, the eyebrows, body hair, the beard, the scalp and/or head hair, or any other cutaneous area of the body. More particularly, the keratin materials according to the invention are the skin and/or the scalp.

The term “skin” is intended to mean all of the skin of the body, and preferably the skin of the face, neckline, neck, arms and forearms, or more preferably still the skin of the face, in particular of the forehead, nose, cheeks, chin and area around the eyes.

The compositions according to the present invention are preferably cosmetic compositions. For the purposes of the present invention, the term “cosmetic” is intended to denote a composition compatible with the skin, mucous membranes and skin appendages.

A composition according to the invention is suitable for application to keratin materials, in particular topical application to the skin, and thus comprises a physiologically acceptable medium, i.e. a medium that is compatible with the skin.

It is preferably a cosmetically acceptable medium, that is to say a medium which exhibits a pleasant color, odor and feel and which does not cause any unacceptable discomfort, that is to say stinging, tautness or redness, liable to dissuade the user from applying this composition.

The expression “at least one” is equivalent to “one or more”.

The expressions “comprises from . . . to . . . ”, “formed from . . . to . . . ”, and “ranging from . . . to . . . ” shall be understood as limits included. Conversely, the expression “between . . . and . . . ” must be understood limits excluded, for the purposes of the present invention.

### DETAILED DESCRIPTION

The use and therefore also the composition according to the invention comprise, in a cosmetically and/or dermatologically acceptable medium, a combination of perlite, pumice stone, microcrystalline cellulose, and cellulose, as defined below.

### Perlite

The composition according to the invention comprises perlite particles. The perlites that may be used in the context of the invention may be expanded perlites. They are generally aluminosilicates of volcanic origin and they have the following composition: 70.0-75.0% by weight silica SiO₂; 12.0-15.0% by weight aluminum oxide Al₂O₃; 3.0-5.0% sodium oxide Na₂O; 3.0-5.0% potassium oxide K₂O; 0.5-2% iron oxide Fe₂O₃; 0.2-0.7% magnesium oxide MgO; 0.5-1.5% calcium oxide CaO; 0.05-0.15% titanium oxide TiO₂. The perlite is milled, dried and then calibrated in a first step. The product obtained, known as perlite ore, is gray-colored and has a size of about 100 μm.

The perlite ore may then be expanded (1000° C./2 seconds) to give more or less white particles. When the temperature reaches 850-900° C., the water trapped in the structure of the material evaporates and brings about the expansion of the material, relative to its original volume. The expanded perlite particles that may be used in the context of the invention may be obtained via the expansion process described in U.S. Pat. No. 5,002,698.

According to one particular embodiment of the invention, the expanded perlite particles used are milled; in this case, they are known as Expanded Milled Perlite (EMP). They preferably have a particle size defined by a median diameter D50 ranging from 0.5 to 300 μm, preferably from 20 to 280 μm and even more preferentially from 100 to 250 μm.

The median diameter D50 or D50 of the particles is measured at 25° C. by laser diffraction with a Malvern Mastersizer instrument. The D50 of a powder corresponds to the value of the particle size that divides the examined population of particles exactly in half.

In other words, in the composition according to the invention, 50% of the exfoliating particles have a size of less than 500 μm, preferably less than 400 μm, preferably less than 300 μm. The D50 of less than 300 μm, preferably from 50 to 280 μm, of the exfoliating particles of the composition according to the invention contributes to obtaining an exfoliation which is at the same time effective, uniform and gentle.

The D50 can be measured according to the standard ISO 9276-parts 1 to 6: “Representation of results of particle size analysis”. In the present description, a laser particle size analyzer, for example of the Malvern Mastersizer type, with software of the RT Sizer type, is used to obtain the particle size distribution of the powder and to deduce therefrom the D50.

By way of examples, mention may be made of the perlites sold under the names Imercare 270P-Scrub, Imercare 400P-Scrub or Imercare 230P Scrub OR by Imerys.

According to an advantageous embodiment, the perlite used according to the invention is unexpanded.

According to another alternative embodiment, the perlite used according to the invention is at least partially expanded.

The perlite particles are generally present in the composition according to the invention in contents ranging from 0.1% to 10%, preferably from 0.5% to 5%, preferably from 0.8% to 3% of perlite, by weight relative to the total weight of the composition, notably in dispersed (aqueous and/or fatty) phase.

In the case of a dry composition, notably in powder form, without aqueous phase or fatty phase in the composition, the perlite content is proportionally higher, and may, for example, represent a content ranging from 10% to 40% by weight out of 100% of dry formula.

### Pumice Stone

The composition according to the invention also comprises pumice stone particles.

Pumice stone, the INCI name of which is pumice, is of volcanic origin. It is formed at temperatures from about 500 to 600° C. from lava projected into the air, which cools on falling, and the degassing of which leads to the formation of bubbles, resulting in a low density and high porosity.

Pumice stone is formed from fragments of rhyolite, of dacite or of andesite. It is considered to be a glass since it does not have a crystalline structure.

Pumice stone particles are abrasive solid particles. In particular, they may have a hardness of greater than or equal to that of the hair, ranging from 3 to 10 Moh, or even greater than or equal to 4, for example greater than or equal to 5, in particular ranging from 5 to 5.5 on the Moh scale.

The pumice stone particles may have a number-median diameter D50 of less than or equal to 500 μm, preferably between 50 and 500 μm, better less than or equal to 300 μm, for example between 100 and 300 μm.

Depending on the range of particles used, the number-median diameter D50 can be determined by using screens or by laser particle size analysis.

It may in particular involve a pumice stone powder sold under the name Ponce O ½ D by Eyraud, with a D50 of approximately 140 μm measured by laser diffraction.

It may also involve a decontaminated pumice stone powder sold under the reference 0-D Ponce by Eyraud, with a D50 of less than 125 μm, or alternatively a pumice stone powder sold under the reference 2B D by Eyraud, with a number-median diameter ranging from 100 to 500 μm.

Mention may be made very particularly of the decontaminated pumice stone powder sold under the reference Ponce O ½ D Ponce Décontaminée by Eyraud, or else Pumice Powder O ½ by Techfil.

The composition according to the invention, in particular when it is in dry form, in powder form, may comprise pumice stone particles in a content ranging from 0.1% to 40% by weight, notably from 1% to 35% by weight, in particular from 5% to 30% by weight, for example from 10% to 25% by weight relative to the total weight of the composition.

When the pumice stone particles are dispersed, for example in the aqueous phase of a composition according to the invention, the content is instead from 0.1% to 8% by weight, preferably from 0.3% to 3%, preferably from 0.7% to 2%, by weight, out of 100% of composition.

### Cellulose

The cosmetic composition according to the invention also comprises cellulose.

Cellulose is a natural polymer of D-glucose, in which the bond between glucose units is of β-1,4 type. Cellulose has the following chemical structure:

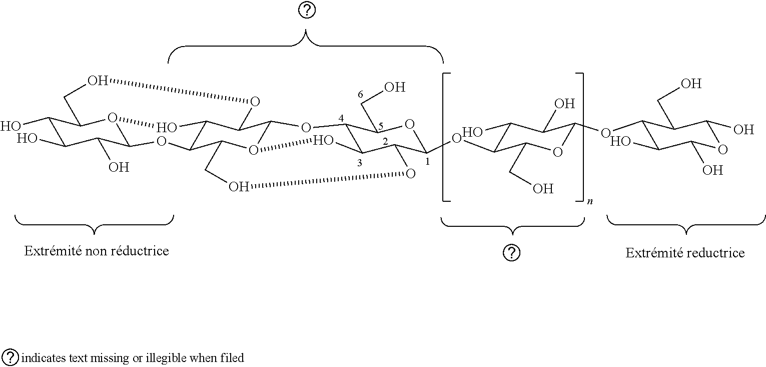

For the purposes of the present invention, said cellulose, other than the microcrystalline cellulose defined below, is an unmodified cellulose, in particular a chemically unmodified cellulose, preferably a natural cellulose.

The composition according to the invention, in particular when it is in dry form, in powder form, preferably comprises cellulose in a content ranging from 0.1% to 40% by weight relative to the total weight of the composition. Of course, the content is expressed in terms of active material. On the other hand, if it is dispersed in an aqueous or fatty phase, the cellulose content is preferably from 0.1% to 6%, preferably from 0.2% to 2%, preferably from 0.2% to 1%, preferably from 0.5% to 1% by weight relative to the total weight of the composition.

### Microcrystalline Cellulose

The cosmetic composition according to the invention comprises microcrystalline cellulose.

The main source of microcrystalline cellulose is plant fiber. The cellulose present therein is a cell wall component in the form of bundles of microfibrils. One portion of these microfibrils is composed of amorphous cellulose, while a second portion is constituted of crystalline cellulose (a polymer is said to be amorphous when its chains are tangled in any way, whereas a polymer of which the chains are aligned into ordered crystals is said to be crystalline).

The other main components of the cell wall are hemicellulose and lignin. Microcrystalline cellulose is thus isolated from the plant fibers, first by separating it from the lignin and hemicellulose, and then by purifying it by acid hydrolysis so as to remove the amorphous cellulose part.

Microcrystalline cellulose is generally prepared from wood pulp: said pulp is cut into small particles which are chemically hydrolyzed, in particular with a mineral acid, then a filtration and drying step takes place.

Microcrystalline cellulose is sold in particular by FMC Biopolymers under the name Avicel or by Vivapur.

Preferably, the microcrystalline cellulose used according to the invention has a particle size defined by a number-median diameter D50 ranging from 80 to 250 μm, preferably from 100 to 200 μm. Preferably, the microcrystalline cellulose is chosen from a microcrystalline cellulose wherein the number-median diameter is less than 180 μm and is preferably 150 μm. The median diameter is a parameter that can be measured by laser particle size analysis or by selective screening.

Preferably, the microcrystalline cellulose is present in a content ranging from 0.1% to 40% by weight relative to the total weight of the composition, when it is in the dry powder form. Of course, the content is expressed in terms of active material. According to the invention, notably if it is dispersed in an aqueous or fatty phase, the microcrystalline cellulose content is preferably from 0.1% to 6%, preferably from 0.2% to 2%, preferably from 0.2% to 1% of microcrystalline cellulose, preferably from 0.5% to 1% by weight relative to the total weight of the composition.

### Composition According to the Invention

In the use and composition according to the invention, the weight ratio of pumice stone to microcrystalline cellulose is greater than or equal to 1, preferably greater than 1. Preferably, the weight ratio of pumice stone to microcrystalline cellulose is between 1 and 3, preferably between 1 and 2, preferably between 1.5 and 2.

Advantageously, the weight ratio of the total content of said two celluloses to the pumice stone content is greater than 1, preferably between 1 and 2.

Preferably, the weight ratio of perlite to pumice stone is greater than 1, preferably between 1 and 2.

A composition in accordance with the invention comprises a combination of several exfoliating filler(s), in particular according to one or more specific contents; preferably, at least some of these fillers are spheroidal, these fillers preferably having a number-median diameter D50 ranging from 100 to 250 μm.

Advantageously, in the composition according to the invention, the perlite and/or the pumice stone and/or the microcrystalline cellulose and/or the cellulose is (are) in the form of particles with a median diameter D50 of less than 300 μm, preferably less than 250 μm, preferably between 100 and 250 μm.

The term “number-median diameter” of the particles is understood to mean the median diameter on 50% by number of the particles (D [0.5]) obtained using a laser diffraction particle size analyzer (e.g. Mastersizer 2000 from the Malvern Company).

Preferably, the perlite and/or the pumice stone and/or the microcrystalline cellulose and/or the cellulose is (are) in the form of particles having a narrow particle size distribution, preferably less than 150 μm, preferably less than 100 μm.

Advantageously, the perlite and/or the pumice stone and/or the microcrystalline cellulose and/or the cellulose is (are) in the form of particles of homogeneous shape, preferably of non-angular shape, preferably of spheroidal shape; preferably, at least said perlite is of homogeneous, preferably non-angular, preferably spheroidal shape; preferably, at least said perlite and the two celluloses are of homogeneous, preferably non-angular, preferably spheroidal shape.

The term “spheroidal” is intended to mean that the particle has a sphericity index, i.e. the ratio between its largest diameter and its smallest diameter, of less than 1.5.

The invention applies to any type of composition and in particular to aqueous compositions, water-in-oil or oil-in-water emulsions, to anhydrous compositions such as pastes or cast products, to aqueous gels or lipophilic gels and to aqueous or lipophilic liquid compositions.

According to a particularly advantageous embodiment, the composition of the invention is in the form of an exfoliating powder and/or a scrubbing powder and/or a cleansing powder.

According to a preferred embodiment, the composition according to the invention comprises an aqueous phase.

### Aqueous Phase

According to a particular embodiment, the composition in accordance with the invention includes an amount of water of at least 40% by weight, preferably ranging from 40% to 95% by weight and better from 50% to 90% by weight, relative to the total weight of the composition.

The water used can be sterile demineralized water and/or a floral water, such as rose water, cornflower water, chamomile water or lime blossom water, and/or a natural thermal or mineral water, such as, for example: Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-saunier water, Eaux-Bonnes water, Rochefort water, Saint-Christau water, Les Fumades water, Tercis-les-Bains water and Avène water. The aqueous phase can also comprise reconstituted thermal water, that is to say a water containing trace elements, such as zinc, copper, magnesium, and the like, reconstituting the characteristics of a thermal water.

The aqueous (or hydrophilic) phase of the composition according to the invention may also contain any water-soluble or water-dispersible additive. Water-soluble additives that may notably be mentioned are polyols comprising from 3 to 10 carbon atoms. The term “polyol” should be understood as meaning any organic molecule including at least two free hydroxyl groups.

Advantageously, the composition of the invention also comprises at least one polyol having from 3 to 10 carbon atoms, preferably from 2 to 6 carbon atoms, such as glycerol, diglycerol, propylene glycol, isoprene glycol, dipropylene glycol, butylene glycol, hexylene glycol, 1,3-propanediol, pentylene glycol, and mixtures thereof, preferably it is glycerol.

Water-soluble additives that may also be mentioned include primary alcohols, i.e. an alcohol including from 1 to 6 carbon atoms, such as ethanol and isopropanol. It is preferably ethanol. The addition of such an alcohol may notably be suitable when the composition according to the invention is used as a product for the body or the hair.

The content of water-soluble or water-dispersible additives in the composition of the invention may range, for example, from 0% to 50% by weight, preferably from 0.5% to 30% by weight and even more preferentially from 2% to 20% by weight, relative to the total weight of the composition.

According to an advantageous embodiment of the invention, the composition has a pH of greater than or equal to 5, which makes it possible in particular to optimize its stability.

The compositions of the invention may contain adjuvants normally used in the cosmetics field and especially those used in cleansing products. Examples of adjuvants that may be mentioned include fragrances, preservatives, pigments, nacres, mineral or organic, matt-effect, whitening or exfoliating fillers, soluble dyes, sunscreens, cosmetic or dermatological active agents such as water-soluble or liposoluble vitamins, antiseptics, antiseborrheic agents, antimicrobials, such as benzoyl peroxide, salicylic acid, triclosan, azelaic acid, and also optical brighteners, nonionic polymers, such as polyvinylpyrrolidone (PVP), anionic polymers, conditioning amphoteric polymers such as polyquaternium products, for instance Polyquaternium-47 sold under the reference Merquat 2001 by Nalco, and fatty substances that are incompatible with the aqueous medium, for instance oils and waxes. The amounts of these various adjuvants are those conventionally used in the field under consideration, for example from 0.01% to 25% of the total weight of the composition. These adjuvants and their concentrations must be such that they do not modify the property desired for the composition of the invention.

Active agents that may be mentioned include any care or cleansing active agent usually used in cosmetics, and in particular antibacterial agents such as octopirox and triclosan, keratolytic agents such as salicylic acid, lactic acid or glycolic acid, salicylic acid derivatives such as 5-n-octanoylsalicylic acid, essential oils, fruit waters (for example from apple or grape) or floral waters (for example rose water), plant extracts (especially from tea, mint, orchid or soybean), leaf, flower or petal inclusions, mineral salts (for example zinc or copper salts), vitamins such as vitamin C (ascorbic acid), vitamin A (retinol), vitamin E, vitamin PP (niacinamide) and vitamin B3 (panthenol), and derivatives thereof.

As fillers, mention may be made of:
additional mineral fillers such as clays other than those defined above,
additional organic fillers other than those defined above, or fibers such as, for example, cellulose or “rayon” fibers (Rayon Flock Rcise NOOO3 MO4® sold by Claremont Flock Corporation).

According to a particular embodiment, the composition according to the invention contains, as fillers, additional exfoliating particles that will allow scrubbing of the skin. Use may be made, as exfoliating particles, of exfoliating or scrubbing particles of mineral, plant or organic origins. Thus, it is possible to use, for example, the shells of crushed fruit kernels such as the crushed apricot kernels or nut shells; sawdust; glass beads; alumina (aluminum oxide) (INCI name: Alumina), for instance the product sold under the name Dermagrain 900 by Marketech International; sugar crystals; beads that melt when applied to the skin, for instance the spheres based on mannitol and cellulose sold under the name Unispheres by Induchem, agar-based capsules sold under the name Primasponge by Cognis, and spheres based on jojoba esters sold under the name Floraspheres by Floratech; diatom frustules such as those sold by Alban Muller under the reference Diatami 60/200 microns.

Advantageously, the composition according to the invention comprises, preferably in an aqueous phase:
0.1% to 10%, preferably from 0.5% to 5%, preferably from 0.8% to 3% of perlite,
0.1% to 8%, preferably from 0.3% to 3%, preferably from 0.7% to 2% of pumice stone,
0.1% to 6%, preferably from 0.2% to 2%, preferably from 0.2% to 1%, preferably from 0.5% to 1% of microcrystalline cellulose,
0.1% to 6%, preferably from 0.2% to 2%, preferably from 0.2% to 1%, preferably from 0.5% to 1% of cellulose, in particular unmodified cellulose;
   the contents being by weight relative to the total weight of the composition representing 100%.

Advantageously, the composition according to the invention is in the form of a composition intended to be rinsed off, such as a cleansing and/or exfoliating and/or scrubbing composition and/or a care mask for keratin materials, preferably the skin, in particular the face, neck and/or scalp, said composition preferably being a 3-in-1 product forming an exfoliating cleansing mask.

Advantageously, the composition according to the invention also comprises at least one foaming surfactant, preferably chosen from anionic surfactants. The anionic foaming surfactant optionally present is chosen in particular from alkyl sulfates, alkyl ether sulfates, isethionates, amino acid derivatives, alkyl glutamates, such as sodium lauroyl glutamate, and mixtures thereof, and preferably chosen from alkyl ether sulfates, such as sodium lauryl ether sulfate. The anionic foaming surfactant(s) are present in concentrations ranging from 0.5% to 30%, preferably from 0.5% to 15% by weight of active material, preferably from 1% to 10% by weight, and more preferentially from 1% to 5% by weight relative to the total weight of the composition.

Advantageously, the composition according to the invention also comprises at least one additive chosen from: hydrophilic active agents, lipophilic active agents, fragrances, preservatives, dyestuffs (pigments or dyes), solvents (lower alcohols), clays or alternatively texturing agents such as pulverulent agents other than the exfoliating particles.

The composition according to the invention may also constitute a composition for caring for oily skin and/or the scalp, in particular when it contains a specific cosmetic active agent for caring for oily skin, such as, for example, azelaic acid, triclosan, piroctone olamine or niacinamide (vitamin PP).

A subject of the present invention is also the cosmetic use of a composition according to the invention, as a cleansing product and/or as an exfoliating agent and/or as a care mask and/or as a 2-in-1 product or a 3-in-1 product, the latter two products respectively comprising 2 or 3 of said possible uses for the same composition.

A subject of the invention is also a method for exfoliating and/or scrubbing and/or massaging and/or cleansing the skin of the body, neck and/or face.

Despite the small size of the particles, the composition of the invention provides an effective scrub without an aggressive effect, because of the particular ratios, shapes and particle sizes of the particles used. In addition to these advantages, the composition of the invention is extremely soft on application.

Another advantage of the above particles is that they can be used in a smaller amount than the conventionally used particles because of their combination in specific ratios, specific particle shapes and their specific particle size.

In the compositions of the invention, from 0.1% to 10% by weight of particles, better from 0.3% to 5% and better still from 0.3% to 1.5%, relative to the total weight of the composition, are preferably used.

As a result, the products of the invention may be used, on a daily basis, as mild cleansing or exfoliating agents or even as facial and/or body massage products. By virtue of the particles selected according to the invention and their mechanical action, the composition of the invention confers a more effective massage than oils or fatty substances, without particles, generally used for massage.

The composition of the invention may be a powder, an aqueous or oily dispersion, an aqueous or oily gel, a water-in-oil or oil-in-water emulsion, a paste or an anhydrous cast product or a product with a high fat content. In summary, the composition of the invention may be provided in any galenic form optionally containing an aqueous phase and/or a fatty phase, the latter “fixing” the particles at the surface of the skin and preventing their removal during massage.

For example, the composition may contain from 5% to 99.9% by weight of fatty and/or aqueous phase, relative to the total weight of the composition, or even from 10% to 50% by weight. The amount of these phases depends in fact on the galenic form.

The optional oils and the fatty or waxy compounds which can be used in the invention may be of mineral, plant, synthetic, silicone or fluorinated origin. They are those conventionally used in the cosmetic and/or dermatological field.

By way of examples, mention may be made, as mineral oil, of paraffin oil or liquid petroleum jelly; as plant oil, of sweet almond, avocado, castor, olive, blackcurrant seed, palm or wheat germ oil, or shea butter; as synthetic oil, of caprylic-capric acid triglycerides, octyldodecanol, cetyl alcohol, stearic or stearyl acid and fatty esters such as butyl myristate or isopropyl or octyl palmitate, or cetearyl octanoate and isopropyl myristate; as silicone oil, of cyclomethicones or polydimethylsiloxanes.

Mention may be made, as waxy compounds which can be used in the invention, of paraffin, jojoba oil and carnauba wax.

To obtain a gel, it is necessary to use hydrophilic or lipophilic gelling agents.

As gelling agents, mention may be made of modified or unmodified clays (bentones) and metal salts of fatty acids (aluminum stearate, ammonium lauryl sulfate); cellulose derivatives (modified celluloses), polycarboxyvinyl polymers and fatty alcohols.

In the case of an emulsion, surfactants may be used to disperse the fats in water to produce oil/water emulsions or, conversely, to disperse the water in the fats to produce water/oil emulsions.

As surfactants, fatty acid esters of glycerol or polyethylene glycol (glyceryl stearate or polyethylene glycol stearate) or of sugar (sorbitan stearate) may be used.

The compositions of the invention may, in addition, contain various ingredients conventionally used in the cosmetic and/or dermatological fields. These ingredients may be constituted of hydrophilic active agents, lipophilic active agents, fragrances, preservatives, dyestuffs (pigments or dyes), solvents (lower alcohols), clays or alternatively texturing agents such as pulverulent agents other than the exfoliating particles. These additional ingredients are used in the usual amounts.

As hydrophilic active agents, mention may be made of polyols such as propylene glycol, glycerol or sorbitol. As lipophilic active agents, mention may be made of essential oils or makeup-removing oils, such as 2-ethylhexyl palmitate, ultraviolet-absorbing screening agents and vitamins.

A subject of the invention is also a method for cleansing and/or scrubbing the skin of the body and/or the face, including the neck, consisting in applying to the skin a composition as defined above, in massaging the skin with this composition to remove dead cells and then in rinsing the skin, usually with water, to remove these dead cells and the particles.

A subject of the invention is also the use of the composition defined above, for cleansing and/or scrubbing the skin of the face, neck and/or body of a human being.

A subject of the invention is also a use of the combination of particles according to the invention in a cosmetically and/or dermatologically acceptable medium, for cleansing and/or scrubbing the skin of the face, neck and/or body of a human being, these particles having a particle size D50 preferably of between 80 μm and 300 μm.

Cleansing of the human body involves the legs as well as the arms, back and stomach.

Given the non-irritating properties of the composition of the invention, it can be used daily, even by individuals with very dry skin.

The invention is illustrated in greater detail by the examples presented below. Unless otherwise indicated, the amounts are given as a percentage % by weight and with respect to active material.

### EXAMPLES

### Example 1

The formulas of table 2 below were tested by a panel of 15 volunteers (laboratory).

Test protocol: Application of 1 g of product to the back of the hand. Spreading on dry skin then the addition of water to cause foaming. Rinsing and drying.

The exfoliating ingredients used in the formulas are listed in table 1:

TABLE 1

| INCI name | Trade name | D50 | |
|---|---|---|---|
| Perlite | IMERCARE 230P SCRUB OR | 150 to 250 | μm |
| | by IMERYS | | |
| Microcrystalline | VIVAPUR CS 150 R5 JRS | 150 | μm |
| cellulose + cellulose: | | | |
| Microcrystalline | VIVAPUR CS 310 R / | 310 | μm |
| cellulose + cellulose | RETTENMAIER | | |
| Microcrystalline | VIVAPUR CS 450 R5 JRS | 450 | μm |
| cellulose + cellulose | | | |
| Pumice | PONCE 0 1/2 D PONCE | | |
| | DECONTAMINEE EYRAUD | | |
| | PUMICE POWDER 0 1/2 | | |
| | TECHFIL | | |

The formulas tested are given in table 2 below.

TABLE 2

| | D50 | Ex | Ex | Ex | Ex | Ex | Ex | | | | | |
|---|---|---|---|---|---|---|---|---|---|---|---|---|
| Exfoliant: | (μm) | 1 | 2 | 3 | 4 | 5 | 6 | Ex7 | Ex8 | Ex9 | Ex10 | Ex11 |
| Perlite | 150 | 1 | 1 | 1 | 1 | 1 | 1 | 1 | 1 | 1 | 1 | 1 |
| | to | | | | | | | | | | | |
| | 250 | | | | | | | | | | | |
| Microcrystalline | 150 | | | | | | | 0.25 + 0.25 | 0.4 + 0.4 | 0.5 + 0.5 | 0.6 + 0.6 | 0.75 + 0.75 |
| cellulose | | | | | | | | | | | | |
| + | | | | | | | | | | | | |
| cellulose | | | | | | | | | | | | |
| Microcrystalline | 310 | 0.5 | 1 | 1.5 | | | | | | | | |
| cellulose + | | | | | | | | | | | | |
| cellulose | | | | | | | | | | | | |
| Microcrystalline | 450 | | | | 0.5 | 1 | 1.5 | | | | | |
| cellulose + | | | | | | | | | | | | |
| cellulose | | | | | | | | | | | | |
| Pumice | | 0.8 | 0.8 | 0.8 | 0.8 | 0.8 | 0.8 | 0.8 | 0.8 | 0.8 | 0.8 | 0.8 |
| Conclusion: | | Scrubbing | | | | | | Scrubbing | Scrubbing | Scrubbing | Scrubbing | Scrubbing |
| | | + | | | | | | + | + | ++++ | +++ | ++++ |
| | | Sensory | | | | | | Sensory | Sensory | Sensory | Sensory | Sensory |
| | | − − | | | | | | + − | + − | ++++ | + − | + −− |

### Conclusion:

From the above tests, it has been demonstrated that the scrubbing system which is both the most effective for the exfoliating (or scrubbing) effect and the most pleasant for the sensory effect comprises a mixture of cellulose and microcrystalline cellulose, a perlite and a pumice stone (or pumice), preferably with a preferential D50 and a preferential amount described above according to the invention.

### Example 2—3-in-1 Composition-Exfoliating Cleansing Mask

The exfoliating (scrubbing) system of Ex 9 was in particular evaluated in the composition (Ex12) of a 3-in-1 exfoliating cleansing mask formula, in table 3 below.

TABLE 3

| INCI name | |
|---|---|
| (Trade name) | Ex12 |
| Water | q.s. 100 |
| XANTHAN GUM | 0.30 |
| ACRYLATES/C10-30 ALKYL ACRYLATE | 0.29 |
| CROSSPOLYMER (Carbopol Ultrez 21) | |
| CARBOMER | |
| (Carbopol 980) | |
| Sodium laureth sulfate | 1.40 |
| Kaolin | 22.00 |
| (IMERCARE 04K) | |
| ZEA MAYS (CORN) STARCH | 4.00 |
| (BEAUTE-BY-ROQUETTE ST005) | |
| Exfoliant of Ex 9 | 1 + 0.8 + 0.50 + 0.50 |
| (Perlite + pumice + cellulose + | |
| microcrystalline cellulose) | |
| 24 h Control: | Texture compliant, |
| Appearance | exfoliating |
| | efficiency +++ |
| Sensoriality | OK |
| pH | 5.00 |
| Viscosity (M4) | 18 UD |
| Viscosity in Poises | 43.00 |
| Conclusion | Compliant - BUT test + |

### Conclusion:

The aqueous composition according to the invention Ex12 makes it possible to obtain a stable, effective formula with a 3-in-1 power (exfoliating cleansing mask). The combination of perlite, pumice stone, a cellulose (unmodified) and a microcrystalline cellulose gives the formula Ex12 exfoliating (scrub/scrubbing) effectiveness.

The compositions according to the invention were well tolerated by all types of skin, even sensitive skin, as attested by the consumer test of example 3 below. They also did not pose an ophthalmic risk.

### Example 3—Consumer Test

### Consumer Test Protocol on Formula Ex12 in Table 3:

TABLE 4

| Method: | Application of the product under normal conditions of use and self-evaluation by | |
|---|---|---|
| | users. | |
| Method and | Test duration: 4 weeks. | |
| duration of | Frequency of application: once a day as a cleanser/scrub and twice a week as a | |
| use of the | mask. | |
| product: | Application sites: on the face. | |
| | Instructions for use: “You will use this product on your face once a day as a | |
| | cleanser/scrub and twice a week as a mask, for 28 days, instead of your usual | |
| | cleanser, scrub and mask.” | |
| | Action: | |
| | Cleanser/scrub: Once a | Mask: |
| | day on wetted skin with a | Twice a week on dry skin, leave on to act for 5 min, wet, |
| | light and gentle massage, | massage lightly and rinse thoroughly. |
| | then rinse thoroughly. | |

| | |
|---|---|
| Inclusion | Women (105) and men (30) living in France, aged 18 to 60. |
| criteria: | Have mixed, normal-oily or oily facial skin. |
| | 50% sensitive skin. |
| | Female phototypes: I & II (35) - III & IV (35) - V & VI (35). No phototype quota |
| | for men. |
| | Have a dull complexion, imperfections (uneven, rough skin, and dilated pores), |
| | superficial marks “marks of old spots, redness, scars . . . ”, skin prone to acne |
| | (blackheads and spots). |
| | Users of cleanser, scrub and cleansing mask if possible. |
| | Men agree to use the tested product every day as a cleanser/scrub and twice a |
| | week as a mask (not necessarily regular users). |
| | Available for the study. |

### Ex12/Observations Obtained

TABLE 5
Texture:

| | |
|---|---|
| Appropriate | 21.7 |

Application:

| | |
|---|---|
| Fresh feeling | 36.4 |
| Easy to apply | 30.1 |
| Pleasant to apply | 19.6 |
| Easy/difficult to rinse off | 12.6 |
| Effectiveness: | |
| Deep cleansing | 29.4 |
| Soft skin | 15.4 |
| Effective | 14 |
| Light skin | 13.3 |
| Acceptability: | |
| Gentle, friendly to the skin | 16.1 |
| Like the existing 3-in-1 products | 10.5 |

Note:
Only statements >10% are listed

1. The cosmetic use of a combination of perlite, pumice stone, microcrystalline cellulose, and cellulose as an exfoliating and/or scrubbing and/or cleansing agent for keratin materials, notably the skin, in particular the face, neck, body and/or scalp.

2. A cosmetic composition, in particular a cleansing and/or exfoliating and/or scrubbing composition for keratin materials, in particular the skin, comprising a combination of perlite, pumice stone, microcrystalline cellulose, and cellulose, wherein the weight ratio of pumice stone to microcrystalline cellulose is greater than or equal to 1, preferably greater than 1.

3. The composition as claimed in claim 2, wherein a weight ratio of pumice stone to microcrystalline cellulose is between 1 and 3.

4. The composition as claimed in claim 2, wherein a weight ratio of total content of said two celluloses to pumice stone content is greater than 1.

5. The composition as claimed in claim 2, wherein a weight ratio of perlite to pumice stone is greater than 1.

6. The composition as claimed in claim 2, wherein the perlite and/or the pumice stone and/or the microcrystalline cellulose and/or the cellulose is (are) in the form of particles with a number-median diameter D50 of less than 300 μm.

7. The composition as claimed in claim 2, wherein the perlite and/or the pumice stone and/or the microcrystalline cellulose and/or the cellulose is (are) in the form of particles having a particle size distribution of less than 150.

8. The composition as claimed in claim 2, wherein the perlite and/or the pumice stone and/or the microcrystalline cellulose and/or the cellulose is (are) in the form of particles of homogeneous shape.

9. The composition as claimed in claim 2, wherein said cellulose other than the microcrystalline cellulose is an unmodified cellulose.

10. The composition as claimed in claim 2, characterized in that it comprises:
0.1% to 10% of perlite,
0.1% to 8% of pumice stone,
0.1% to 6% of microcrystalline cellulose,
0.1% to 6% of cellulose;
the contents being by weight relative to the total weight of the composition representing 100%.

11. The composition as claimed in claim 2, characterized in that it is in the form of an exfoliating powder and/or a scrubbing powder and/or a cleansing powder.

12. The composition as claimed in claim 2, characterized in that it is in the form of a composition intended to be rinsed off.

13. The composition as claimed in claim 2, characterized in that it also comprises at least one foaming surfactant.

14. The composition as claimed in claim 13, wherein the at least one anionic foaming surfactant is chosen from alkyl sulfates, alkyl ether sulfates, isethionates, amino acid derivatives, alkyl glutamates, and mixtures thereof.

15. The composition as claimed in claim 13, wherein the anionic foaming surfactant(s) are present in a concentration from 0.5% to 30%, preferably from 0.5% to 15% by weight of active material, preferably from 1% to 10% by weight, and more preferentially from 1% to 5% by weight relative to the total weight of the composition.

16. The composition as claimed in claim 2, also comprising at least one polyol having from 3 to 10 carbon atoms.

17. The composition as claimed in claim 2, also comprising at least one additive chosen from: hydrophilic active agents, lipophilic active agents, fragrances, preservatives, dyestuffs (pigments or dyes), solvents (lower alcohols), clays or alternatively texturing agents such as pulverulent agents other than the exfoliating particles.

18. The cosmetic use of a composition as claimed in claim 2, as a cleansing product and/or as an exfoliating product and/or as a care mask and/or as a 2-in-1 product or as a 3-in-1 product, the latter two products comprising respectively 2 or 3 of said abovementioned uses for one and the same composition.
